(19) United States
(12) Patent Application Publication
(10) Pub. No.: US 2019/0156955 A1
(43) Pub. Date: May 23, 2019

(54) IDENTIFYING PROGRAM MEMBER DATA RECORDS FOR TARGETED OPERATIONS

(71) Applicant: Palantir Technologies Inc., Palo Alto, CA (US)

(72) Inventors: Michael Winlo, Palo Alto, CA (US); Dustin Janatpour, San Mateo, CA (US); Kyle Erickson, Cupertino, CA (US); Aaron Tucker, Silver Spring, MD (US)

(21) Appl. No.: 16/255,727

(22) Filed: Jan. 23, 2019

Related U.S. Application Data
(63) Continuation of application No. 15/811,446, filed on Nov. 13, 2017, now abandoned.
Application No. 15/811,446 is a continuation of application No. 14/958,855, filed on Dec. 3, 2015, now abandoned.
(60) Provisional application No. 62/212,512, filed on Aug. 31, 2015.

(51) Int. Cl. G16H 50/30; G16H 80/00; G16H 15/00

(52) U.S. Cl. CPC G16H 50/30; G16H 15/00; G16H 80/00

(57) ABSTRACT

A system comprising a computer-readable storage medium storing at least one program and a method for identifying care management program members to target for therapeutic intervention are presented. In example embodiments, the method includes identifying a set of candidate member data records from a plurality of member data records, and using data models to determine a risk score, a benefit score, and a participation score associated with each candidate member data record. The benefit score provides a measure of a propensity of the corresponding member to benefit from the therapeutic intervention program, and the participation score provides a measure of a propensity of the corresponding member to participate in the therapeutic intervention program. The method further includes identifying target members for the therapeutic intervention program from the set of candidate member data records based on respective benefit scores and participation scores.

### RELATED APPLICATION

This application is a continuation of, and claims the benefit of U.S. patent application Ser. No. 15/811,446, filed on Nov. 13, 2017, which is a continuation of, and claims the benefit of U.S. patent application Ser. No. 14/958,855, entitled “IDENTIFYING PROGRAM MEMBER DATA RECORD S FOR TARGETED OPERATIONS”, filed on Dec. 3, 2015, which claims the benefit of priority to U.S. Provisional No. 62/212,512, entitled “IDENTIFYING CARE MANAGEMENT PROGRAM MEMBERS TO TARGET FOR THERAPEUTIC INTERVENTION”, filed on Aug. 31, 2015, all of which are incorporated by reference herein in its entireties.

### TECHNICAL FIELD

The subject matter disclosed herein relates to database management and computer networks. In particular, example embodiments relate to systems and methods for analyzing a plurality of data records stored on a networked database to identify a subset of data records for targeted operations.

### BACKGROUND

Care management programs are designed to assist members to manage medical, social, and mental health conditions more effectively by engaging in a collaborative process with care providers that seeks to improve clinical outcomes, reduce utilization of care services, and lower costs. In some instances, care management programs involve providing therapeutic interventions to individuals whose behavioral health problems (e.g., depression or alcoholism) are interfering with the management of their chronic physical health conditions (e.g., diabetes, or COPD).

Traditionally, members of the care management programs are selected for therapeutic intervention programs by identifying members with either known or suspected clinical or behavioral health conditions who exhibit evidence they are managing physical health conditions poorly. In conventional practice, identification of these members is based on these members having certain known or suspected conditions and high monthly costs associated with care to address such conditions. However, these crude, high cost only cut-offs typically ignore important member-level differences in engagability, utilization patterns, imp actability of the program, reimbursement, and risk adjustment as well as other important health data (e.g., pharmaceutical claims, or health risk data). Furthermore, these methodologies only assess the member's current health status whereas it is often more important to prevent future high utilization. As a result, conventional methodologies often result in unintelligent member selection with a large number of qualified and high risk members not entering the care management program and large numbers of temporarily high cost members entering the program that may overwhelm limited clinical staff In addition to the inefficient utilization of human resources, these conventional methodologies often result in an over utilization of computational and network resources due to the generation and transmission of large amounts of data associated with electronic correspondence (e.g., email) used to engage with members who are unlikely to respond and thus require rep eat correspondence that causes further strain to the computational and network resources.

### BRIEF DESCRIPTION OF THE DRAWINGS

Various ones of the appended drawings merely illustrate example embodiments of the present inventive subject matter and cannot be considered as limiting its scope.

FIG. 1 is an architecture diagram depicting a data processing platform having a client-server architecture configured for exchanging data, according to an example embodiment.

FIG. 2 is a block diagram illustrating various modules comprising a target member identification service, which is provided as part of the data processing platform, consistent with some embodiments.

FIG. 3 is a block diagram illustrating a benefit data model, consistent with some embodiments.

FIG. 4 is a block diagram illustrating a participation data model, consistent with some embodiments.

FIG. 5 is a flowchart illustrating a method for identifying members of a care management program to target for a therapeutic intervention program, according to some embodiments.

FIG. 6 is a flowchart illustrating a method for identifying candidate member data records, according to some embodiments.

FIG. 7 is a flowchart illustrating a method for determining a benefit score, according to some embodiments.

FIG. 8 is a flowchart illustrating a method for determining a participation score, according to some embodiments.

FIG. 9 is a flowchart illustrating a method for determining risk scores associated with a member population, according to some example embodiments.

FIG. 10 is an interface diagram illustrating a user interface for defining a member cohort and presenting analytics related thereto, according to example embodiments.

FIG. 11 is a diagrammatic representation of a machine in the example form of a computer system within which a set of instructions for causing the machine to perform any one or more of the methodologies discussed herein may be executed.

### DETAILED DESCRIPTION

Reference will now be made in detail to specific example embodiments for carrying out the inventive subject matter. Examples of these specific embodiments are illustrated in the accompanying drawings, and specific details are set forth in the following description in order to provide a thorough understanding of the subject matter. It will be understood that these examples are not intended to limit the scope of the claims to the illustrated embodiments. On the contrary, they are intended to cover such alternatives, modifications, and equivalents as may be included within the scope of the disclosure.

Aspects of the present disclosure relate to systems and methods for identifying program (e.g., a care management program) members to target for therapeutic intervention. In example embodiments, the system identifies qualifying candidates from a member pool based on the members' available health data (e.g., claims, health risk assessments, and clinical notes), utilization patterns of health care services and deviations from expected costs. The system scores qualifying candidates according to their propensity to benefit from a therapeutic intervention program. The scored propensity of a care management program member to benefit from therapeutic intervention conveys a likelihood that therapeutic intervention will result in a change to the pattern of health care utilization of the member such that the costs associated with the member are reduced. An example of a benefit from therapeutic intervention is a reduction in the member's number of yearly visits to an emergency room. The system scores a member's propensity to benefit based on clinical conditions of the member as well as other behavioral and social factors.

The system further scores qualifying candidates according to their propensity to participate in the therapeutic intervention program. The propensity of a care management program member to benefit from therapeutic intervention refers to the likelihood that the member will participate in the therapeutic intervention. The system scores a member's scored propensity to participate in therapeutic intervention based, for example, on demographic information and social factors.

The system uses the scored propensities to benefit and propensities to participate to identify candidate members to target for the therapeutic intervention program (referred to herein as “target members”). Consistent with some embodiments, the system may further match identified target candidates with health care staff of the therapeutic intervention program based on staff availability and staff skills (e.g., language).

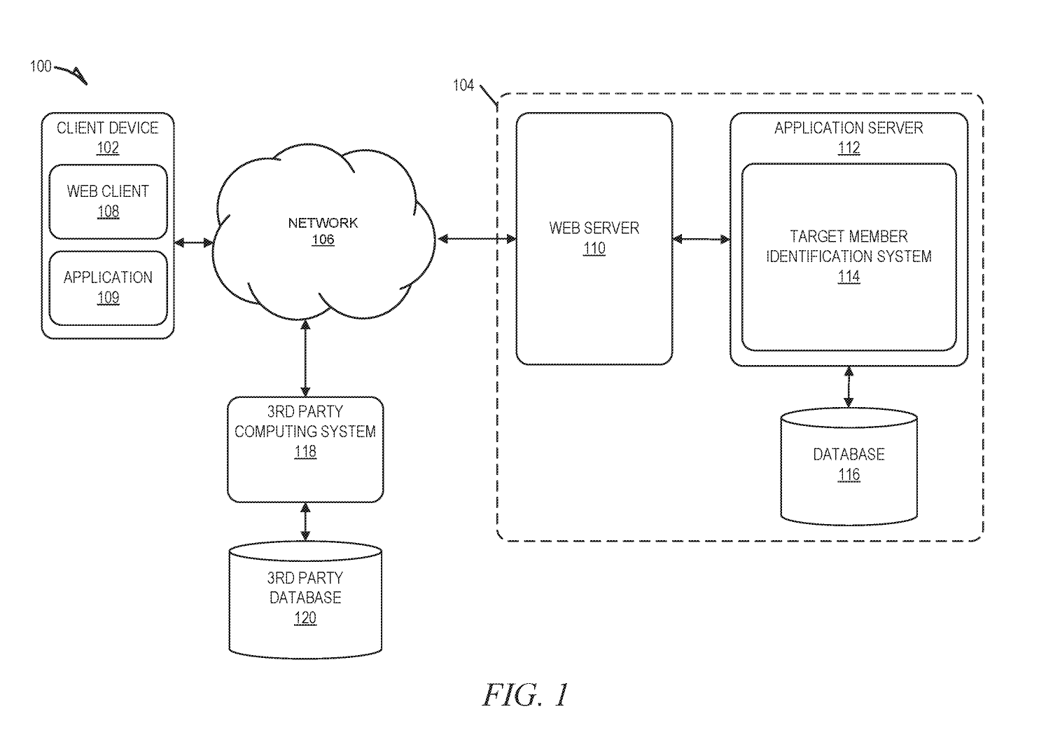

FIG. 1 is an architecture diagram depicting a network system 100 having a client-server architecture configured for exchanging data, according to an example embodiment. While the network system 100 shown in FIG. 1 employs a client-server architecture, the present inventive subject matter is, of course, not limited to such an architecture, and could equally well find application in an event-driven, distributed, or peer-to-peer architecture system, for example. Moreover, it shall be appreciated that although the various functional components of the network system 100 are discussed in the singular sense, multiple instances of one or more of the various functional components may be employed.

As shown, the network system 100 includes a client device 102 in communication with a data processing platform 104 over a network 106. The data processing platform 104 communicates and exchanges data with the client device 102 that pertains to various functions and aspects associated with the network system 100 and its users. Likewise, the client device 102, which may be any of a variety of types of devices that include at least a display, a processor, and communication capabilities that provide access to the network 106 (e.g., a smart phone, a tablet computer, a personal digital assistant (PDA), a personal navigation device (PND), a handheld computer, a desktop computer, a laptop or netbook, or a wearable computing device), may be operated by a user (e.g., a person) of the network system 100 to exchange data with the data processing platform 104 over the network 106.

The client device 102 communicates with the network 106 via a wired or wireless connection. For example, one or more portions of the network 106 may comprise an ad hoc network, an intranet, an extranet, a Virtual Private Network (VPN), a Local Area Network (LAN), a wireless LAN (WLAN), a Wide Area Network (WAN), a wireless WAN (WWAN), a Metropolitan Area Network (MAN), a portion of the Internet, a portion of the Public Switched Telephone Network (PSTN), a cellular telephone network, a wireless network, a Wireless Fidelity (Wi-Fi) network, a Worldwide Interoperability for Microwave Access (WiMax) network, another type of network, or any suitable combination thereof.

In various embodiments, the data exchanged between the client device 102 and the data processing platform 104 may involve user-selected functions available through one or more user interfaces (UIs). The UIs may be specifically associated with a web client 108 (e.g., a browser) or an application 109, executing on the client device 102, and in communication with the data processing platform 104.

Turning specifically to the data processing platform 104, a web server 110 is coupled to (e.g., via wired or wireless interfaces), and provides web interfaces to, an application server 112. The application server 112 hosts one or more applications (e.g., web applications) that allow users to use various functions and services of the data processing platform 104. For example, the application server 112 may host a target member identification system 114 that is used to identify qualifying members of a care management program to target for therapeutic intervention. In some embodiments, the target member identification system 114 runs and executes on the application server 112, while in other embodiments, the application server 112 provides the client device 102 with a set of instructions (e.g., computer-readable code) that causes the web client 108 of the client device 102 to execute and run the target member identification system 114.

The target member identification system 114 analyzes various data to identify qualifying candidates for therapeutic intervention based on the members' utilization patterns and deviations from expected costs. The target member identification system 114 determines a propensity of each candidate to benefit from a therapeutic intervention program, and a propensity of each candidate to participate in the therapeutic intervention program. The target member identification system 114 uses the determined propensities to benefit and propensities to participate to identify candidate members to target for the therapeutic intervention program. The target member identification system 114 may further be used to match identified target candidates with health care staff based on staff availability and staff skills (e.g., language).

The data analyzed by the target member identification system 114 includes member data that comprises a plurality of member records. Each member record includes information about a member (e.g., a person) of a care management program. For example, each member record includes a member identifier (e.g., a name), demographic information (e.g., age, gender, and ethnicity), clinical conditions that the member has (e.g., behavioral, mental, or physical health conditions), and utilization data. The utilization data includes information related to the member's utilization of health care services. Accordingly, the utilization data may, for example, include types of health care services utilized, dates health care services where utilized, and costs associated with the utilization of health care services. The member data can also be provided by clinicians and other individuals interacting with the member.

The data analyzed by the target member identification system 114 (e.g., member data) is obtained from a third-party computing system 118 (e.g., corresponding to a care management provider), and in particular, a third-party database 120 communicatively coupled to the third-party computing system 118. The data may be routinely, automatically retrieved (e.g., nightly) by the target member identification system 114, or manually provided by a user of the third-party computing system 118 or the client device 102 for subsequent processing and analysis by the target member identification system 114.

The data obtained from the third-party computing system 118 is stored in a database 116, which is a machine-readable storage medium that is communicatively coupled to the application server 112 (e.g., via wired or wireless interfaces). The data processing platform 104 may further include a database server (not shown) that facilitates access to the database 116. The database 116 may include multiple databases that may be internal or external to the data processing platform 104. Data representative of the analytics and other pertinent data generated by the target member identification system 114 are also stored on the database 116.

The database 116 also stores staff data that includes information related to staff members of the care management program. The staff data includes a plurality of staff member data records, each of which corresponds to a staff member of the care management program. Each staff member data record includes information describing a particular staff member. Individual staff member data records may, for example, include a staff member identifier, demographic data, information about staff member skills (e.g., languages spoken), and staff availability data (e.g., information about staff member schedules and workloads).

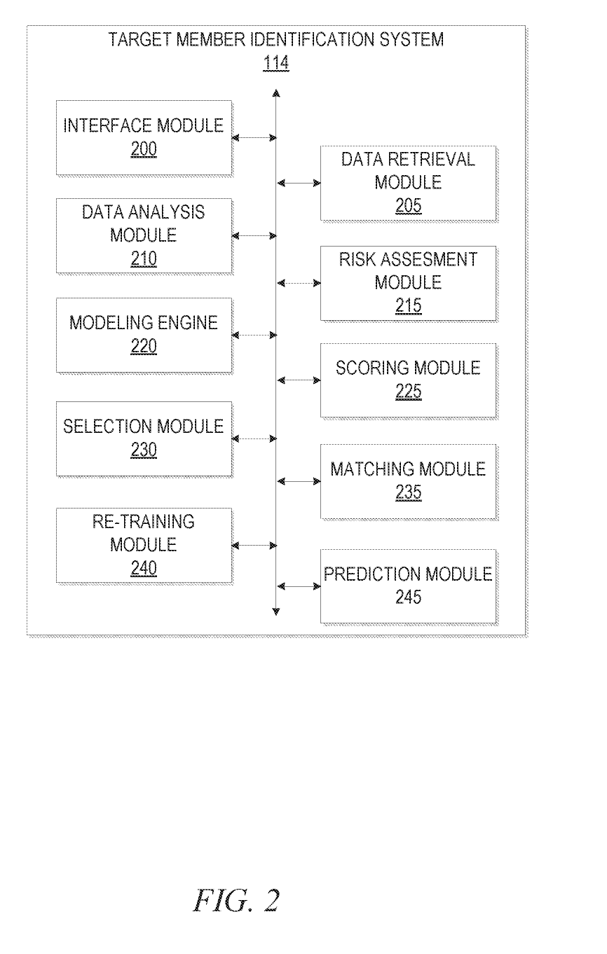

FIG. 2 is a block diagram illustrating various modules comprising the target member identification system 114, which is provided as part of the data processing platform 104, consistent with some embodiments. As is understood by skilled artisans in the relevant computer and Internet-related arts, the modules and engines illustrated in FIG. 2 represent a set of executable software instructions and the corresponding hardware (e.g., memory and processor) for executing the instructions. To avoid obscuring the inventive subject matter with unnecessary detail, various functional components (e.g., modules and engines) that are not germane to conveying an understanding of the inventive subject matter have been omitted from FIG. 2. However, a skilled artisan will readily recognize that various additional functional components may be supported by the target member identification system 114 to facilitate additional functionality that is not specifically described herein. Furthermore, the various functional modules and engines depicted in FIG. 2 may reside on a single computer (e.g., a client device), or may be distributed across several computers in various arrangements such as cloud-based architectures.

The target member identification system 114 is shown as including an interface module 200, a data retrieval module 205, a data analysis module 210, a risk assessment module 215, a modeling engine 220, a scoring module 225, a selection module 230, a matching module 235, a re-training module 240, and a prediction module 245 all configured to communicate with each other (e.g., via a bus, shared memory, a switch, or application programming interfaces (APIs)). The aforementioned modules of the target member identification system 114 may, furthermore, access one or more databases that are part of the data processing platform 104 (e.g., database 116), and each of the modules may access one or more computer-readable storage media of the client device 102.

The interface module 200 receives requests from various client devices, and communicates appropriate responses to the requesting client devices. The interface module 200 may receive requests from client devices in the form of Hypertext Transfer Protocol (HTTP) requests, or other web-based, application programming interface (API) requests. For example, the interface module 200 provides a number of interfaces (e.g., APIs or user interfaces that are presented by the client device 102) that allow data to be received by the target member identification system 114.

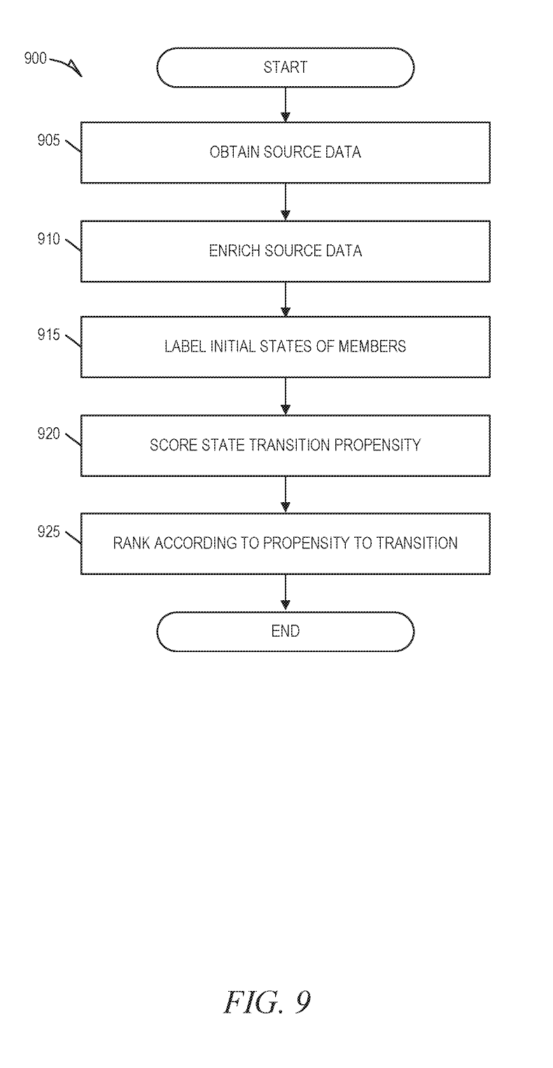

The interface module 200 also provides user interfaces that include graphical representations of various analytics and other output produced by the target member identification system 114. These user interfaces may include various graphs, charts, and other graphics used, for example, to represent members of a care management program and their various attributes. The interface module 200 also receives and processes user input received through such user interfaces. An example of the user interfaces provided by the interface module 200 is discussed below in reference to FIG. 9.

The data retrieval module 205 is configured to retrieve data for processing and analysis. For example, the data retrieval module 205 obtains member data comprising a plurality of member records corresponding to members of a care management program, and staff data comprising a plurality of staff member data records. In some embodiments, the data retrieval module 205 retrieves such data from the third-party database 120 of the third-party computing system 118 through appropriate requests (e.g., API requests or calls) transmitted over the network 106. The data may be retrieved by the data retrieval module 205 on a periodic basis (e.g., nightly). In some embodiments, the data retrieval module 205 obtains data from a location specified by a user (e.g., via a user interface provided by the interface module 200).

The data analysis module 210 is configured to analyze data to develop various analytics related to members of care management programs. For example, the data analysis module 210 analyzes member data to identify characteristics of a particular member cohort. The member cohort may constitute a subset of a total member population of a care management program. The member cohort may be defined based on user constraints (e.g., input by a user using an interface provided by the interface module 200). The user constraints defining the member cohort may, for example, include an age range of members, total cost range of individual members, various utilization measures (e.g., number of emergency room (ER) visits), derived metrics (e.g., a threshold for a deviation from expected cost value) or various combinations thereof.

Given a set of user constraints defining a member cohort, the data analysis module 210 accesses a corpus of member data corresponding to member data records that fulfill the user constrains, and the data analysis module 210 determines a number of analytics for the member cohort including total monthly costs of the members, possibly preventable costs (e.g., based on mappings in academic literature), deviation from expected costs, (e.g., based on a deviation of actual costs to expected cost), prior engagement of the member with a care management program, a percent of member cohort that is male, a percent of member cohort that is female, percent of members younger than 65 years old, or health facility utilizations (e.g., ER visits, in-patient stays, PCP visits).

The data analysis module 210 is also configured to analyze data to identify qualifying candidates for a therapeutic intervention program. The qualifying candidates may be identified from an entire candidate pool or may be identified from a specific member cohort, which may be defined based on member age and cost, for example. The data analysis module 210 identifies the qualifying candidates through analysis of member data that comprises data records for each member.

Each member data record contains information about the member including for example, a member identifier, demographic data about the member, the member's clinical conditions, and utilization data of the member. The demographic data includes information describing characteristics of the member such as gender, age, ethnicity, location (e.g., hometown or current location), employment history, and education history, among others. The utilization data includes information related to the member's utilization of health care services; accordingly, the utilization data may, for example, include types of health care services utilized, dates health care services where utilized, and costs associated with the utilization of health care services.

In identifying qualifying candidates, the data analysis module 210 determines an expected cost value for each member based on the demographic data and clinical condition information contained in respective member data records. The data analysis module 210 calculates the expected cost value of each member based on estimates of costs associated with treating the member's clinical condition(s), which may vary based on demographic information. For example, the cost of treating an elder person for diabetes may be less than treating an adolescent for diabetes. In calculating the expected cost of each member, the data analysis module 210 may access a look-up table (e.g., stored in the database 116) that includes estimated costs values associated with each possible type of clinical condition. The estimated costs values may be based on historical utilization data of members with the corresponding clinical condition(s).

The data analysis module 210 further determines an actual cost of each member included in the member cohort. The data analysis module 210 determines the actual cost value associated with each member based on the utilization data contained in respective member data records. The utilization data included in each member data record includes information related to the utilization of health care services by the member and the costs associated therewith.

Accordingly, in determining the actual cost associated with each member, the data analysis module 210 calculates an aggregate of all costs associated with the utilization of health care services by the member.

The data analysis module 210 further calculates the difference between the expected cost and the actual cost associated with each member data record, and the average difference between the expected cost and actual cost for all members. The data analysis module 210 then selects candidate members based on a deviation of the difference between expected costs and actual costs associated with a particular member from an average difference between expected and actual costs. For example, the data analysis module 210 may select members whose actual and expected costs differ by a value above a certain threshold above the average difference.

The risk assessment module 215 is configured to determine and rank each member's propensity to deviate significantly from their expected medical costs over a period of time (e.g., over the next 12 months). The risk assessment module 215 uses logistic regression to compute a risk score for each member that provides a measure for the member's propensity to deviate from expected medical costs over a time period (e.g., over the next 12 months) by more than a threshold amount (e.g., $1250 or more per month). These risk scores can be used as a basis for stratifying members into risk categories (e.g., High, Moderate, and Low) for the purposes of allocating clinical resources and designing coordinated care plans. The risk assessment module 215 incorporates claims data (e.g., combined medical and pharmaceutical claims), coverage data (e.g., coverage and eligibility information), member demographic data (e.g., demographic information provided by the health plan), and health risk assessment (HRA) response data (e.g., responses to HRAs administered to members on behalf of health plans) to arrive at these risk scores. The motivating assumption is that the highest risk members are most likely to benefit from the highest intensity clinical care management resources.

In addition to the above referenced source data (e.g., claims data, coverage data, member demographic data, and HRA response data), the risk assessment module 215 incorporates a number of custom metrics derived from the source data and computed over a lookback period (based on data availability and member eligibility). For example, the risk assessment module 215 may compute: a total member medical cost based on a sum of amounts paid on medical claims; a average per claim amount based on an average amount paid on medical claims; a per member per month medical cost; a total prescription cost per member based on a sum of amounts paid on pharmaceutical claims; an average amount paid on pharmaceutical claims; a per member per month pharmaceutical cost; a per member per month combined medical and pharmaceutical cost; a sum of amounts paid on medical and pharmaceutical claims; a difference (e.g., in dollars) between expected costs (CMS defined capitations rate given a set of hierarchical condition categories (HCCs), adjusted for medical loss ratio (MLR)) and an observed cost; a population average difference between expected cost and observed cost; a population stand deviation of difference between expected cost and observed cost; per-member deviation from expected costs (in standard deviations) from the population average; a cost factor based on a sum of member's HCC coefficients, which is used to calculate expected cost; a CMS-defined capitation rate, given a set of HCCs and a CMS plan rating of three and a half stars; a MLR based on CMS-defined capitations rate, given a set of HCCs, adjusted for MLR; a sum of ER encounters; a sum of possibly preventable ER costs (in dollars) based academic literature; a sum of in-patient encounters; a list of unique codes for drugs prescribed; a list of unique drug names for drugs prescribed; a list of unique pharmacological classes for drugs prescribed; a list of government schedules for drugs prescribed; a drug count based on a count of unique drugs prescribed; suspected conditions based on mapping of International Classification of Diseases (ICD), Current Procedural Terminology (CPT), and Healthcare Common Procedure Coding System (HCPCS) codes to suspected conditions; and HCCs based on CMS mapping of ICD-9 codes to chronic conditions.

The risk assessment module 215 includes a model that defines risk as the propensity to deviate from expected medical costs over a time period (e.g., over the next 12 months) by more than a threshold amount (e.g., $1250 or more per month). Expected monthly costs include what one would expect a member to cost given their disease burden, and the risk assessment module 215 calculates expected monthly costs by assigning HCCs to members (e.g., based on the ICD-9 codes that appear in a look-back period of claims history), summing up the CMS-defined capitation premium for those HCCs, and adjusting for MLR. The risk assessment module 215 calculates observed monthly costs as per-member per-month medical costs over eligible months during the same lookback period. The risk assessment module 215 calculates deviation from expected cost by subtracting observed monthly costs from expected monthly costs.

High deviation is not a pure cost function, because it is sensitive to what is known about a member's disease burden. Accordingly, the risk assessment module 215 flags people who are unexpectedly costly regardless of the underlying reason for that cost deviation. For example, members may deviate significantly from expected costs because they are high ER utilizers, because of excess in-patient visits, because psychological or social factors are interfering with the management of an otherwise management condition, or because they are “mispriced” due to undiagnosed chronic conditions (member is lacking appropriate HCCs, therefore the plan is being reimbursed at a lower rate).

The risk assessment module 215 leverages all source and derived data elements to generate features as inputs in the model. During risk assessment module 215 binarizes categorical and ordinal variables, and continuous variables are used directly and not normalized.

Members who exhibit high cost or utilization at the time of stratification may be chronically ill and poorly managed, or they may have experienced an acute incident in their recent past that has caused their cost and utilization to spike temporarily. These latter members may regress to the cost and/or utilization mean and may not benefit significantly from care management resources that are designed to benefit chronically ill individuals. The model of the risk assessment module 215 therefore needs to account for likely regression to the mean and treat these members differently from the chronically ill and/or poorly managed.

The risk assessment module 215 addresses the regression to the mean problem by dividing the member population into different groups, or “starting states.” These different groups have different baseline risk profiles, and therefore should be modeled differently. For example, the factors that cause a relatively healthy person to suddenly deviate significantly from their expected costs are likely to be different from those that cause a chronically ill person to experience a similar cost spike. As an example, the risk assessment module 215 may label members as high-deviation, chronic, or non-chronic low-deviation. High-deviation members whose average monthly medical costs exceed their expected monthly medical costs by more than a threshold amount (e.g., $1250 or more). Chronic members are members whose claims history suggest they have one or more high risk chronic conditions, but whose average monthly medical costs do not exceed their expected costs by more than the threshold amount (e.g., $1250 or more). Non-chronic low deviation members are members whose claim history does not indicate they have a high risk chronic condition, and whose average monthly medical costs do not exceed their expected costs by more than the threshold amount (e.g., $1250 or more).

Once the risk assessment module 215 has labeled members with a particular starting state, the risk assessment module 215 trains multiple distinct logistic regression models to cover each state transition. For example, the risk assessment module 215 may a model to cover the following state transitions: high-deviation to high-deviation; high-deviation to chronic; high-deviation to non-chronic low-deviation; chronic to high-deviation; chronic to chronic; chronic to non-chronic low-deviation; non-chronic low-deviation to high-deviation; non-chronic low-deviation to chronic; and non-chronic low-deviation to non-chronic low-deviation. The training set for each model includes a sample of all eligible members. The model training uses L1 regression to address the collinearity between several of the cost metrics by inducing sparseness on the model.

For each member, the risk assessment model 215 generates features over the 12 months preceding and the 12 months following the split date. Split dates were selected according to the following logic, in order to maximize the length of pre- and post-periods for model performance and validation purposes: (1) if members have completed an HRA any time before a predetermined date, the split data is the HRA completion date; (2) if members have completed an HRA any time before the predetermined date, the split date is the predetermined date. The output of each model training cycle is a set of feature weights that can be used to compute scores for each of the state transitions listed above. Feature weights are coefficients in a logistic regression model and can be roughly interpreted as the extent to which each feature contributes to the outcome.

The risk assessment model 215 generates features for members over a lookback period (e.g., of up to 12 months) preceding propensity scoring (depending on data availability and member eligibility). The risk assessment model 215 then computes propensity scores for each member for each of their three possible state transitions. For each state transition, features are weighted according to the most recently trained model. The propensity scores indicate the relative likelihood that a member will move in to, or remain in, a given state within a certain period of time (e.g., up to 12 months).

The risk assessment model 215 ranks the member population by propensity to transition to a high-deviation state within a certain period (e.g., over the next 12 months). In some embodiments, for a given member, one of three state transition models are used for final rank ordering, depending on their starting state: high-deviation to high-deviation; chronic to high-deviation; and non-chronic low-deviation to high-deviation. This ordered population can then be stratified into High, Moderate, and Low risk categories for the purposes of developing coordinated care plans.

The modeling engine 220 is configured to generate data models to facilitate the identification of target members for therapeutic intervention. In particular, the modeling engine 220 is configured to generate a benefit data model and a participation data model. The modeling engine 220 generates these data models through analysis of historical member data.

The benefit data model is used to quantify a potential of a member to benefit from therapeutic intervention, wherein the benefit includes a change to the utilization of health care services that results in an overall reduction in costs associated with the member. For example, a member may be determined to have benefitted from a therapeutic intervention program when the therapeutic intervention program results in: a change to a number of visits the member makes to an emergency room over a fixed period of time; a change to a number of in-patient admissions of the member over a fixed period of time; a change to the number of visits to a PCP over a fixed period of time; a change to the rate at which pharmaceuticals are collected; a change to a per month cost associated with the member; a change in the difference between expected and actual cost associated with the member; or a change in health risk (e.g., a number and severity of known conditions).

Accordingly, in generating the benefit data model, the modeling engine 220 performs feature extraction by applying logistic regression (or other regression techniques) to the historical member data to identify common factors that lead members to benefit from therapeutic intervention in the past. The benefit data model generated by the modeling engine 220 includes one or more benefit metrics that are factors that contribute to propensity of members to benefit from therapeutic intervention. The benefit data model further includes a weight for each benefit metric that indicates the degree to which the metric contributes to the propensity of members to benefit from therapeutic intervention. In some instances, in performing the analysis of the historical member data, the modeling engine 220 may identify certain conditions that are more likely to result in a member receiving a benefit from therapeutic intervention, and certain conditions that are more likely to result in a member not receiving a benefit from therapeutic intervention. The modeling engine 220 may control for confounding or collinear factors by applying L1 regularization or similar techniques.

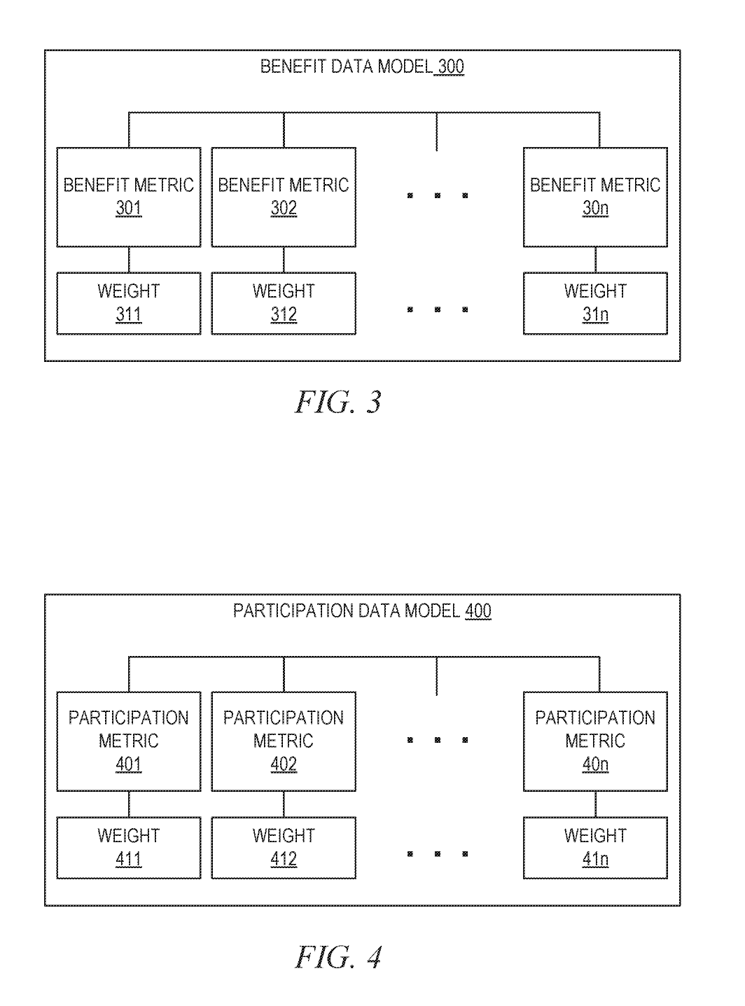

As an example of the benefit data model generated by the modeling engine 220, FIG. 3 is a block diagram illustrating a benefit data model 300, consistent with some embodiments. As shown, the benefit data model 300 includes benefit metrics 301-30n, each of which is a factor that contributes to the propensity of members to benefit from therapeutic intervention. The benefit data model 300 also includes weights 311-31n, each of which corresponds to a respective benefit metric 301-30n, and indicates a degree to which respective benefit metrics contribute to the propensity of members to benefit from therapeutic intervention.

The benefit metrics 301-30n include behavioral and social factors, and factors related to the clinical conditions of the member. The benefit metrics 301-30n may, for example, include a number of visits the member makes to an emergency room over a fixed period of time, a number of in-patient admissions over a fixed period of time, a number of in-patient re-admissions over a fixed period of time, a per month cost associated with the member, a number of primary care physician (PCP) visits, an amount of preventable cost (e.g., the difference between expected and actual cost associated with the member), engagement with medical treatment (e.g., prescription adherence and PCP visit frequency), and a number and severity of known conditions.

Referring back to FIG. 2, the modeling engine 220 is also configured to generate participation data models for use in identifying target members for a therapeutic intervention program. The participation data model is used to quantify likelihood of a member to participate in a therapeutic intervention program. Members who are likely to participate in the therapeutic intervention program are those members who may be expected to be contacted and who are expected to consent to participation.

Similar to the generation of benefit data models (e.g., benefit data model 300), in generating the participation data model, the modeling engine 220 performs feature extraction by applying logistic regression (or other regression techniques) to the historical member data to identify common factors that contributed to members participating in past therapeutic intervention programs. The participation data model generated by the modeling engine 220 includes one or more participation metrics that are factors that contribute to propensity of members to participate in therapeutic intervention. The participation data model further includes a weight for each participation metric that indicates a degree to which the metric contributes to the propensity of members to participate in therapeutic intervention.

As an example of the participation data model generated by the modeling engine 220, FIG. 4 is a block diagram illustrating an example participation data model 400, consistent with some embodiments. As shown, the participation data model 400 includes participation metrics 401-40n, each of which is a factor that contributes to the propensity of members to participate in therapeutic intervention. The participation metrics 401-40n may, for example, include demographic and social factors related to a member's potential for participating in therapeutic intervention. Participation metrics may, for example, include: a number of outreach attempts (e.g., calls, and emails) before member successfully enrolls; time of day calls are attempted; a number of sessions a member completes; a length of time a member stays in an intervention group; and a number of times a staff member interacts with a member. The participation data model 400 also includes weights 411-41n, each of which corresponds to a respective participation metric 401-40n, and indicates a degree to which respective participation metrics 401-40n contributes to the propensity of members to participate in therapeutic intervention.

Referring back to FIG. 2, the scoring module 225 is configured to score members based on a variety of metrics using the data models generated by the modeling engine 220. In particular, the scoring module 225 generates a benefit score and a participation score for each member selected by the data analysis module 210 as a candidate for therapeutic intervention. The benefit score provides a quantified measure of a propensity of a member to benefit from therapeutic intervention. In other words, the benefit score indicates a likelihood that a member will benefit from therapeutic intervention. A change to the pattern of health care utilization of the member that results in a reduction to costs associated with the member is an example of a benefit that may be provided to members by therapeutic intervention.

In scoring members of a member cohort, the scoring module 225 accesses a benefit data model (e.g., benefit data model 300) and a participation data model (e.g., participation data model 400) corresponding to the therapeutic intervention program. The scoring module 225 then determines a value for each metric included in each respective data model based on the member's data record. For example, for a metric corresponding to emergency room visits, the scoring module 225 accesses the utilization data included in the member data to determine a number of visits the member made to the emergency room. The scoring module 225 then weights each determined metric value based on the respective metric weight indicated in the respective data model. The scoring module 225 then aggregates the individual weighted metrics to generate the respective score (e.g., the benefit score and the participation score).

The selection module 230 is configured to identify target members of the care management program for participation in the therapeutic intervention program based on the benefit and participation scores determined by the scoring module 225. In some embodiments, the selection module 230 may identify candidate members as target members based on the risk, participation, or benefit score exceeding a certain threshold value. In some embodiments, the selection module 230 may identify candidate members as target members based on a combination of the risk, benefit, and participation scores exceeding a certain threshold value. In some embodiments, the selection module 230 may identify candidate members as target members based on the combination of the risk, participation, and benefit scores exceeding a certain threshold value.

In some embodiments, the selection module 230 may rank candidate members according to a combination of risk, benefit, and participation scores and select candidate members as target members based on the ranking. For example, the selection module 230 may select the top ranked members (e.g., top 10) as target members for therapeutic intervention. As another example, the candidate members may be rank-ordered by “risk score” until the total population required to fill all available interventions (after factoring in attrition and graduation rates) is reached. Members may then queued for the program for which they score the highest on the propensity to benefit dimension. Members may be queued for more than one program at a time. Engagement efforts may then organized by the propensity to engage score. Some programs may prefer to target difficult to reach members early in the cycle (e.g., high propensity to benefit, low propensity to engage) and then, as the intervention cycle ends or as they reach the end of the year—they switch emphasis to the highest propensity to engage members. Depending on health plan preference, this toggling of engagement propensity, can make it possible for a member to be scored for multiple interventions and ultimately pulled into an intervention for which they have a high propensity to engage, but a relatively lower propensity to benefit.

The matching module 235 is configured to match identified target members with staff members of the care management program to facilitate or assist with the therapeutic intervention program. The matching module 235 matches target members to the staff member based on an analysis and comparison of the corresponding member data record to the staff member data. The matching module 235 analyses the staff member data to identify one or more staff member data records that provide a fit to respective target members in terms of staff skills (e.g., specialist qualifications or training language skills, or particular interest in a certain clinical condition or demographic) and staff availability. The matching module may also consider current staff capacity (e.g., number of active members a staff member can interact with). In this way, target members are matched to suitable staff members based on staff member schedules and skills that may be useful in providing care to target members. As an example, the matching module 235 may match a target member data record to a staff member data record based on the target member's availability in the evenings, and the corresponding staff member being scheduled to work evenings. As another example, the matching module 235 may match a member who can only speak Spanish with a Spanish speaking staff member.

The re-training module 240 is responsible for retraining the risk, benefit, or participation data models based on updated data. To this end, the re-training module 240 may, at routine intervals or when new data becomes available, obtain updated source data and provide to the risk assessment module 215 or modeling engine 220 for up dating the models used therein. In this way, respective data models are kept accurate with the most up-to-date information. Further, the re-training module 240 may configured to modify any one of the data models described herein by incorporating feedback from clinicians interacting with members (e.g., textual feedback enter into a user interface and received via the interface module 200). For example, as clinicians are talking to members and learning about their circumstances, this information can be captured through a user interface and received information can become a new feature incorporated into any one of the models described herein. In this way, clinicians' clinical assessments can be used to inform any one of the methodologies described herein.

The prediction module 245 is configured to predict member diagnosis based on member data. The prediction of member diagnosis includes examining procedures typically linked to a particular diagnosis to establish a set of typical procedures for each diagnosis. The prediction module 245 may then analysis member data of a particular member to determine a probability that the member might be missing a diagnosis based on the fact that they are receiving procedures typically associated with a particular diagnosis.

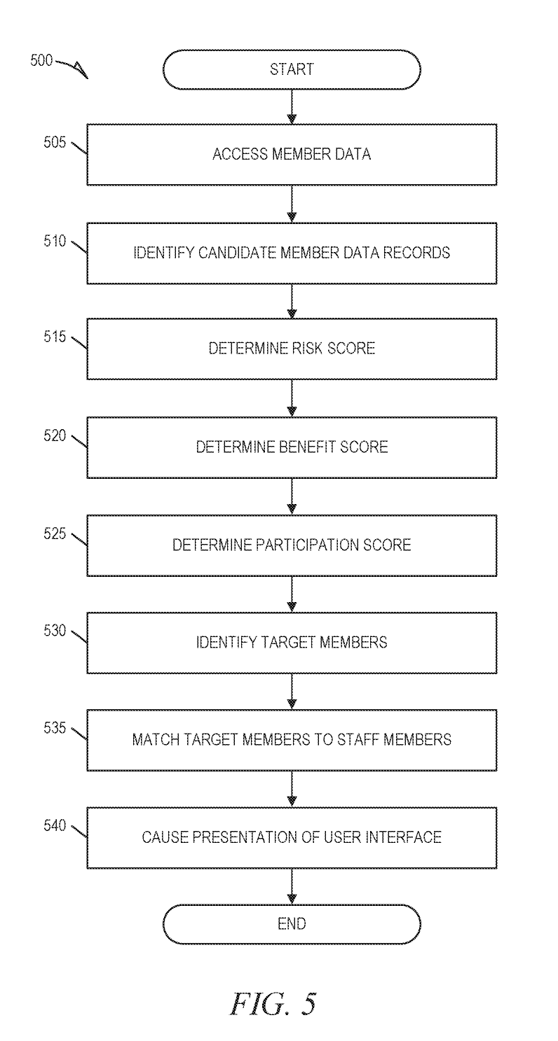

FIG. 5 is a flowchart illustrating a method 500 for identifying members of a care management program to target for a therapeutic intervention program, according to some embodiments. The method 500 may be embodied in computer-readable instructions for execution by one or more processors such that the operations of the method 500 may be performed in part or in whole by the application server 112. In particular, the operations of the method 500 may be performed in part or in whole by the target member identification system 114; accordingly, the method 500 is described below by way of example with reference thereto. However, it shall be appreciated that at least some of the operations of the method 500 may be deployed on various other hardware configurations and the method 500 is not intended to be limited to the application server 112 or the target member identification system 114.

At operation 505, the data analysis module 202 accesses a corpus of member data stored in the database 116. The member data includes a plurality of member data records corresponding to members of a care management program. Each member data record contains information about a respective member of the care management program. In some embodiments, the member plurality of member data records may correspond to a member cohort that may represent a subset of members of a care management program selected based on user defined constraints such as member age and total costs associated with each individual member.

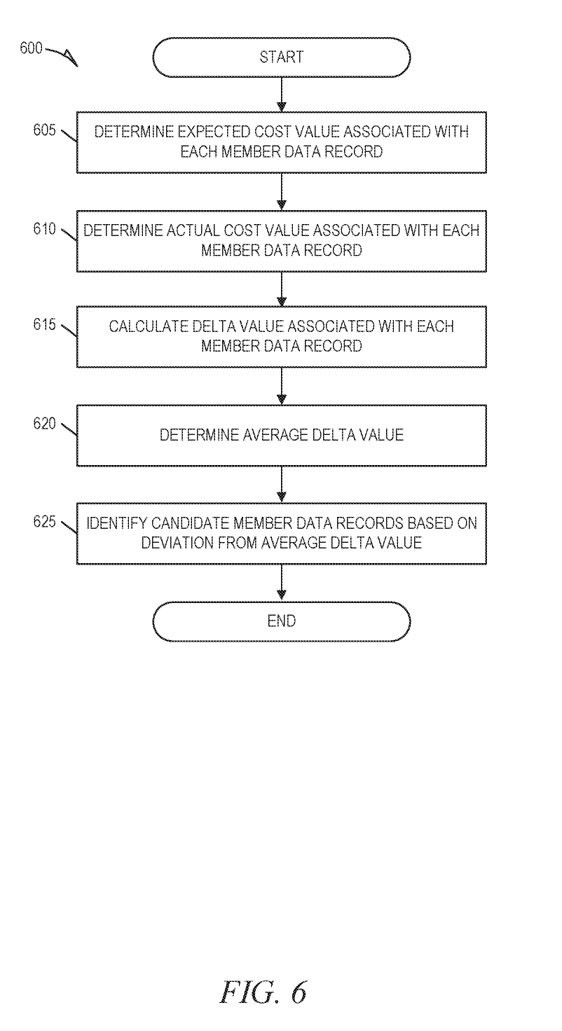

At operation 510, the data analysis module 202 identifies candidate member data records from the plurality of member data records. The candidate member data records correspond to candidate members that qualify for therapeutic intervention. The candidate members are a subset of the plurality of members identified based on a deviation of actual costs associated with each member from the expected costs associated with each member. As an example of the sub-operations involved in operation 510, FIG. 6 is a flowchart illustrating a method 600 for identifying candidate member data records, according to some embodiments. The method 600 may be performed as part of the operation 510 of method 500, consistent with some embodiments. The method 600 may be embodied in computer-readable instructions for execution by one or more processors such that the operations of the method 600 may be performed in part or in whole by the application server 112. In particular, the operations of the method 600 may be performed in part or in whole by the data analysis module 210 of the target member identification system 114; accordingly, the method 600 is described below by way of example with reference thereto. However, it shall be appreciated that at least some of the operations of the method 510 may be deployed on various other hardware configurations and the method 600 is not intended to be limited to the application server 112 or the target member identification system 114.

At operation 605, the data analysis module 210 determines an expected cost value associated with each member data record included in the member data. The data analysis module 210 determines the expected cost value of each member record based on estimated costs associated with the clinical conditions of each corresponding member. Information regarding the clinical conditions of each member are included in the respective member data record of the member. The estimated costs used by the data analysis module 210 are based on historical costs associated with each possible clinical condition, which may be included in a look-up table or other data object accessed by the data analysis module 210.

At operation 610, the data analysis module 210 determines an actual cost value associated with each member data record included in the member data. The data analysis module 210 determines the actual cost value associated with each member data record based on the utilization data included therein. The utilization data included in each member data includes information related to the utilization of health care services by the member and the costs corresponding thereto. Accordingly, in determining the actual cost value associated with each member data record, the data analysis module 210 calculates an aggregate of all costs associated with the utilization of health care services by the member.

At operation 615, the data analysis module 210 calculates a delta value associated with each member data record included in the member data. The data analysis module 210 calculates the delta value associated with each member data record by calculating the difference between the expected cost value and the actual cost value associated with each member data record. In this way, the delta value represents the difference between the actual cost and the expected cost of each member.

At operation 620, the data analysis module 210 calculates an average delta value for the plurality of member data records. The data analysis module 210 calculates the average delta value by taking an average of all delta values associated with the plurality of member data records.

At operation 625, the data analysis module 210 selects candidate member data records from the plurality of member data records based on the deviation of delta values associated with respective member data records from the average delta value of the plurality of member data records. For example, the data analysis module 210 may select the member data records with an associated delta value deviation (the deviation of the delta value associated with a particular member data record with the average data value of all member data records) above a predefined threshold. The predefined threshold may be a default value or a value input by a care management program administrator using an interface provided by the interface module 200. The candidate member data records selected by the data analysis module 210 correspond to the qualifying member candidates for the therapeutic intervention program.

Referring back to FIG. 5, at operation 515, the risk assessment module 215 determines, using one or more risk data models, a risk score associated with each candidate member data record based on the data included therein. The risk score provides a measure of a likelihood that a member will deviate from expected medical costs over a time period (e.g., over the next 12 months) by more than a threshold amount (e.g., $1250 or more per month). The determining of the risk score for each member may include labeling an initial state of the member (e.g., high-deviation, chronic, and non-chronic low-deviation), and using one or more risk data models to compute a propensity score for state transitions of the member. The propensity scores represent the relative likelihood that a member will move into, or remain in, a given state. The member risk scoring may further include ranking the members according to propensity to transition to a high-deviation state within a certain time period (e.g., 12 months). Further details regarding the determination of the risk score associated with each candidate member data record are discussed below in reference to FIG. 9, according to some example embodiments.

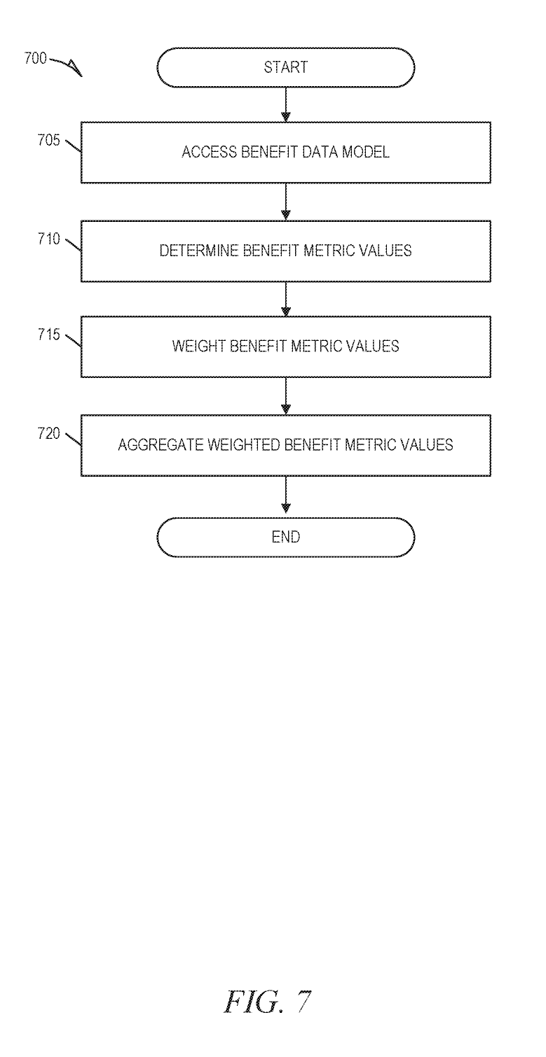

At operation 520, the scoring module 225 determines, using a benefit data model, a benefit score associated with each candidate member data record based on the data included therein. More specifically, the scoring module 225 determines a benefit score for each corresponding candidate member. Individual member benefit scores provide a measure of the propensity of the member to benefit from therapeutic intervention programs. As an example of the operations involved in determining a benefit score for a particular member, FIG. 7 is a flowchart illustrating a method 700 for determining a benefit score, according to some embodiments. Consistent with some embodiments, the method 700 may be performed as part of the operation 520 of the method 500. The method 700 may be embodied in computer-readable instructions for execution by one or more processors such that the operations of the method 700 may be performed in part or in whole by the application server 112. In particular, the operations of the method 515 may be performed in part or in whole by the functional components of the scoring module 225 of the target member identification system 114; accordingly, the method 700 is described below by way of example with reference thereto. However, it shall be appreciated that at least some of the operations of the method 700 may be deployed on various other hardware configurations and the method 700 is not intended to be limited to the application server 112.

At operation 705, the scoring module 225 accesses a benefit data model corresponding to the therapeutic intervention program (e.g., from the database 116). The benefit data model (e.g., benefit data model 300) includes a number of benefit metrics that contribute to the propensity of a member to benefit from the therapeutic intervention program. The benefit metrics include behavioral factors, social factors, and factors related to the clinical conditions of the member. The benefit metrics may, for example, include a number of visits the member makes to an emergency room over a fixed period of time, a number of in-patient admissions over a fixed period of time, a per month cost associated with the member, the difference between expected and actual cost associated with the member, and a number and severity of known conditions.

At operation 710, the scoring module 225 determines benefit metric values for each of the benefit metrics included in the benefit data model based the member data record of the member. For example, if the benefit data model includes a benefit metric corresponding to the number of in-patient admissions, the scoring module 225 accesses the utilization data included in the member data record to determine the number of in-patient admissions of the member. At operation 715, the scoring module 225 weights each benefit metric value according to the respective weight of each benefit metric specified by the benefit data model. At operation 720, the scoring module 225 aggregates the weighted benefit metric values to generate the benefit score for the member of the care management program. For example, the scoring module 225 may calculate the average of the weighted benefit metric values to determine the overall benefit score for the member.

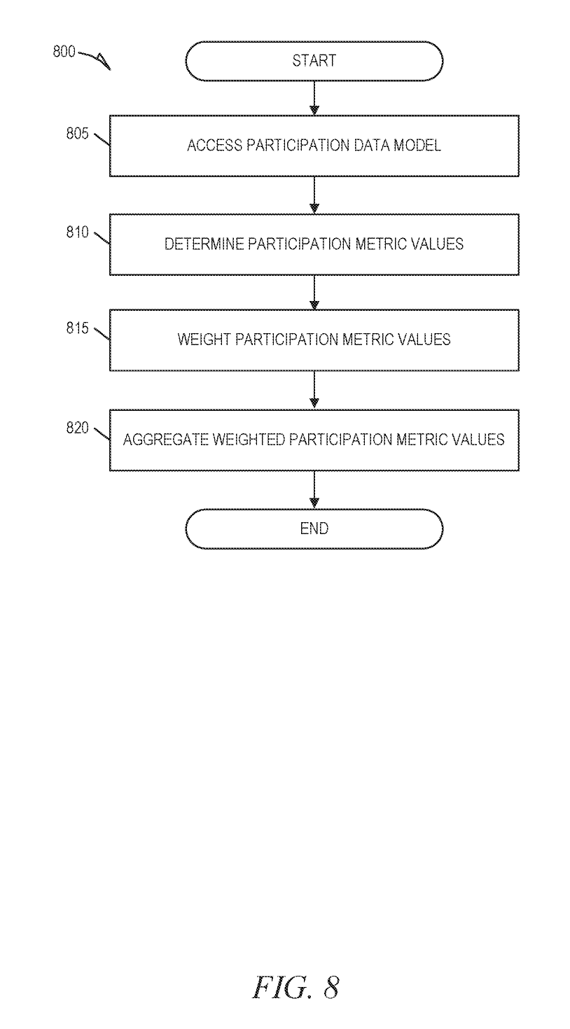

Referring back to FIG. 5, at operation 525, the scoring module 225 calculates, using a participation data model, a participation score associated with each candidate member data record based on the data included therein. More specifically, the scoring module 225 determines a participation score for each corresponding candidate member. Individual member participation scores provide a measure of the propensity of the member to participate in the therapeutic intervention program. As an example of the operations involved in determining a participation score for a particular member, FIG. 8 is a flowchart illustrating a method 800 for determining a participation score, according to some embodiments. Consistent with some embodiments, the method 800 may be performed as part of the operation 520 of the method 500. The method 800 may be embodied in computer-readable instructions for execution by one or more processors such that the operations of the method 800 may be performed in part or in whole by the application server 112. In particular, the operations of the method 800 may be performed in part or in whole by the functional components of the scoring module 225 of the target member identification system 114; accordingly, the method 800 is described below by way of example with reference thereto. However, it shall be appreciated that at least some of the operations of the method 800 may be deployed on various other hardware configurations and the method 800 is not intended to be limited to the application server 112.

At operation 805, the scoring module 225 accesses a participation data model corresponding to the therapeutic intervention program (e.g., from the database 116). The participation data model (e.g., participation data model 400) includes a number of participation metrics that contribute to the propensity of a member to participate in the therapeutic intervention program. The participation metrics include demographic and social factors.

At operation 810, the scoring module 225 determines participation metric values for each of the participation metrics included in the participation data model based on the member data record of the member. At operation 815, the scoring module 225 weights each participation metric value according to the respective weight of each participation metric specified by the participation data model. At operation 820, the scoring module 225 aggregates the weighted participation metric values to generate the participation score for the member of the care management program. For example, the scoring module 225 may calculate the average of the weighted participation metric values to determine the overall participation score for the member.

Referring back to FIG. 5, at operation 530, the selection module 230 identifies one or more target members of the care management program for participation in the therapeutic intervention program. The selection module 230 identifies the one or more target members from the candidate members based on respective risk, benefit, and participation scores. In some embodiments, the selection module 230 may identify candidate members as target members based on either the risk, participation, or benefit score exceeding a certain threshold value. In some embodiments, the selection module 230 may identify candidate members as target members based on various combinations of the risk, benefit, and participation scores exceeding a certain threshold value. In some embodiments, the selection module 230 may identify candidate members as target members based on the aggregate of the risk, participation and benefit scores exceeding a certain threshold value. In some embodiments, the selection module 230 may rank candidate members according to combinations of risk, benefit, and participation scores and select candidate members as target members based on the ranking.

At operation 535, the matching module 235 matches each identified target member to a suggested staff member based on the staff member data stored in the database 116. More specifically, the matching module 235 matches member data records corresponding to target members with one or more staff member data records. The matching of member data records to the staff member data records may be based on staff skills and staff availability. In this way, target members may be match to one or more staff members that provide the best fit in terms of scheduling and skills that may be useful in providing care to target members. As an example, the matching module 235 may match a target member data record to a staff member data record based on the corresponding target member being a Spanish speaker, and the corresponding staff member being fluent in Spanish.

At operation 540, the interface module 200 causes presentation of a user interface on the client device 102. The user interface includes information about the plurality of members. For example, the user interface may include an indication of which members are qualifying candidate members, the risk, benefit, and participation scores of the qualifying candidate members, and an indication of the members identified as target members for the therapeutic intervention program. Further, members may be presented in such a way that the features that contributed the most to their various scores are presented. In this way, users such as clinicians actually get useful, and actionable information. For example, if the benefit data model gives a high weight to the clinical feature of “depression” and a member has a history of “depression” then that feature will be presented as a main, contributing factor in the determination of the score.

FIG. 9 is a flowchart illustrating a method for determining risk scores associated with a member population, according to some example embodiments. The method 900 may be embodied in computer-readable instructions for execution by one or more processors such that the operations of the method 900 may be performed in part or in whole by the application server 112. In particular, the operations of the method 900 may be performed in part or in whole by the functional components of the risk assessment module 225 of the target member identification system 114; accordingly, the method 900 is described below by way of example with reference thereto. However, it shall be appreciated that at least some of the operations of the method 900 may be deployed on various other hardware configurations and the method 900 is not intended to be limited to the application server 112.

At operation 905, the risk assessment module 215 obtains source data from one or more network locations (e.g., servers or databases). The source data includes claims data (e.g., combined medical and pharmaceutical claims), coverage data (e.g., coverage and eligibility information), member demographic data (e.g., demographic information provided by the health plan), and HRA response data (e.g., responses to HRAs administered to members on behalf of health plans).

At operations 910, the risk assessment module 215 enriches the source data with derived metrics. The derived metrics used to enrich the source data include, for example, a total member medical cost based on a sum of amounts paid on medical claims; a average per claim amount based on an average amount paid on medical claims; a per member per month medical cost; a total prescription cost per member based on a sum of amounts paid on pharmaceutical claims; an average amount p aid on pharmaceutical claims; a per member per month pharmaceutical cost; a per member per month combined medical and pharmaceutical cost; a sum of amounts paid on medical and pharmaceutical claims; a difference (e.g., in dollars) between expected costs (CMS defined capitations rate given a set of hierarchical condition categories (HCCs), adjusted for medical loss ratio (MLR)) and an observed cost; a population average difference between expected cost and observed cost; a population stand deviation of difference between expected cost and observed cost; per-member deviation from expected costs (in standard deviations) from the population average; a cost factor based on a sum of member's HCC coefficients, which is used to calculate expected cost; a CMS-defined capitation rate, given a set of HCCs and a CMS plan rating of three and a half stars; a MLR based on CMS-defined capitations rate, given a set of HCCs, adjusted for MLR; a sum of ER encounters; a sum of possibly preventable ER costs (in dollars) based academic literature; a sum of in-patient encounters; a list of unique codes for drugs prescribed; a list of unique drug names for drugs prescribed; a list of unique pharmacological classes for drugs prescribed; a list of government schedules for drugs prescribed; a drug count based on a count of unique drugs prescribed; suspected conditions based on mapping of International Classification of Diseases (ICD), Current Procedural Terminology (CPT), and Healthcare Common Procedure Coding System (HCPCS) codes to suspected conditions; and HCCs based on CMS mapping of ICD-9 codes to chronic conditions.

At operation 915, the risk assessment module 215 labels each member with an initial state. As an example, the risk assessment module 215 may label members as high-deviation, chronic, or non-chronic low-deviation. High-deviation members whose average monthly medical costs exceed their expected monthly medical costs by more than a threshold amount (e.g., $1250 or more). Chronic members are members whose claims history suggest they have one or more high risk chronic conditions, but whose average monthly medical costs do not exceed their expected costs by more than the threshold amount (e.g., $1250 or more). Non-chronic low deviation members are members whose claim history does not indicate they have a high risk chronic condition, and whose average monthly medical costs do not exceed their expected costs by more than the threshold amount (e.g., $1250 or more).

At operation 920, the risk assessment module 215 scores each member according to their propensity to transition to each state. For each state transition, features are weighted according to the most recently trained model. The propensity scores indicate the relative likelihood that a member will move in to, or remain in, a given state within a certain period of time (e.g., up to 12 months).

At operation 925, the risk assessment model 215 ranks the member population by propensity to transition to a high-deviation state within a certain period (e.g., over the next 12 months). In some embodiments, for a given member, one of three state transition models are used for final rank ordering, depending on their starting state: high-deviation to high-deviation; chronic to high-deviation; and non-chronic low-deviation to high-deviation. This ordered population can then be stratified into High, Moderate, and Low risk categories for the purposes of developing coordinated care plans.

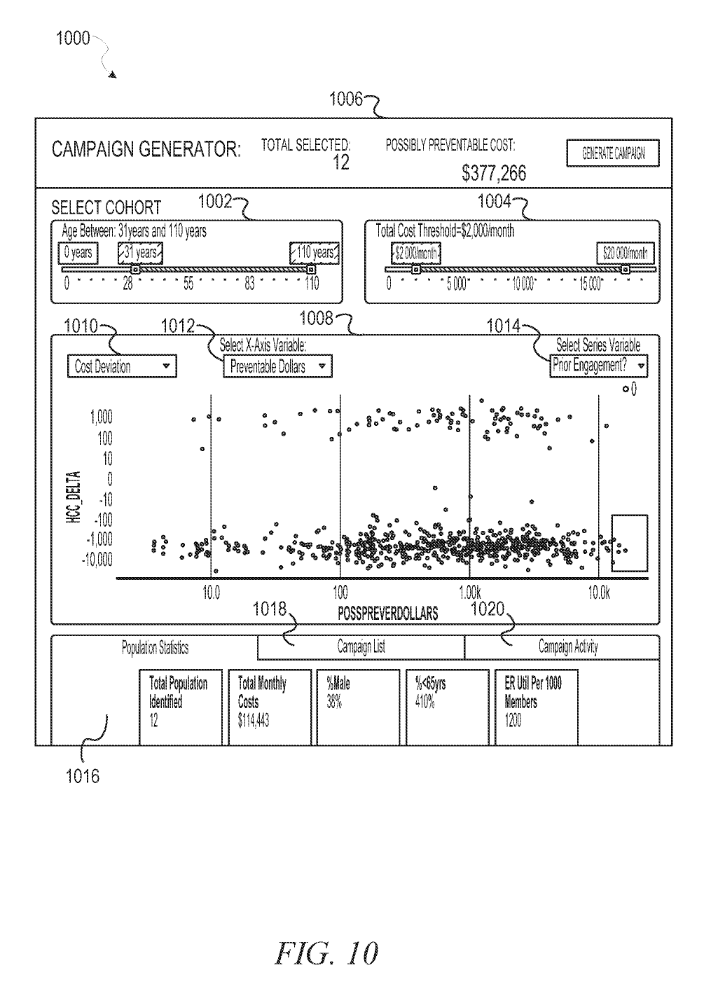

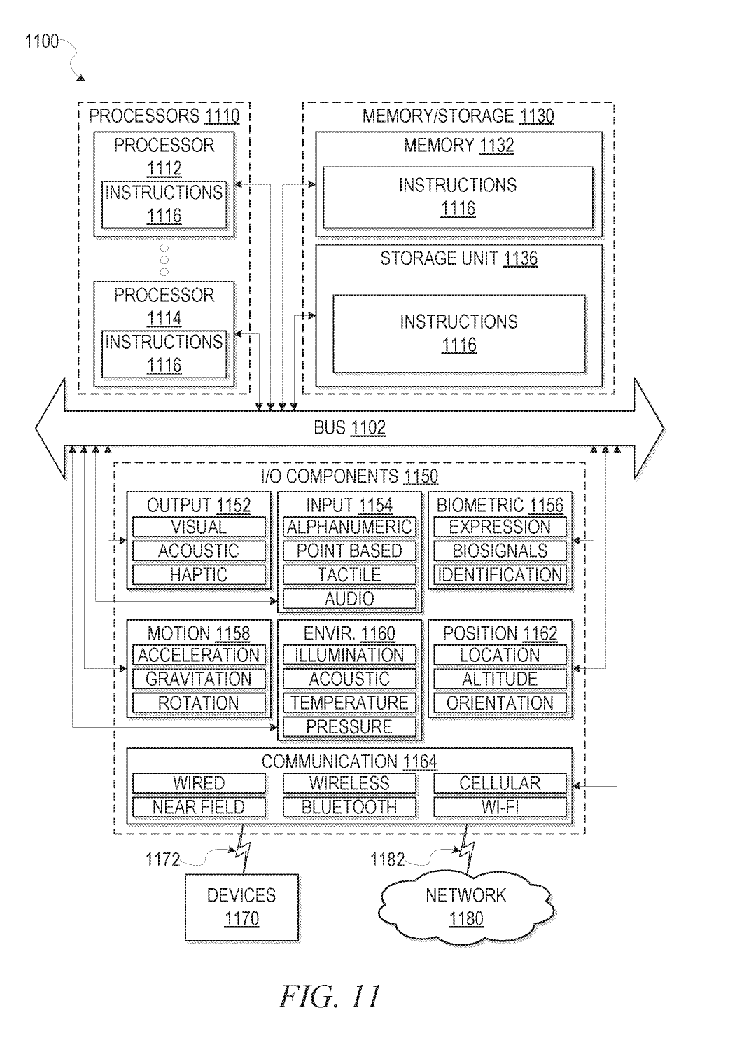

FIG. 10 is an interface diagram illustrating a user interface 1000 for defining a member cohort and presenting analytics related thereto, according to example embodiments. As illustrated in FIG. 10, the user interface 1000 includes interactive elements 1002 and 1004 for selecting a member cohort (e.g., a subset of members of a care management program). Each of the interactive elements 1002 and 1004 is used by a user to specify an aspect of the member cohort. More specifically, the interactive element 1002 is used to specify an age range for members of the member cohort. As shown in this example, the age range for the member cohort is set between 31 years and 110 years. The interactive element 1004 is used to specify a cost range associated with individual members of the member cohort. As shown in this example, the cost range for the member cohort is set between $2,000/month and $20,000/month.

M embers that fulfill the criteria specified using the interactive elements 1002 and 1004 are included in the member cohort. Once such members are identified, the data analysis module 210 analyzes the corresponding member data records to determine statistics about such members. For example, a header 1006 of the user interface 1000 includes information related to the total number of members in the member cohort, and the total possibly preventable cost associated with the member cohort (e.g., the difference between the actual and expected costs associated with the member cohort).

The user interface 1000 also includes a graph 1008 for graphically displaying information about the member cohort. Users may use the drop-down menus 1010, 1012, and 1014 to select and change the information displayed in the graph 1008. For example, drop-down menu 1010 is used to specify the type of data displayed in the graph 1008, which in this example is “Cost Deviation.” The drop-down menus 1012 and 1014 are used, respectively, to select the variables of the X-Axis and Y-Axis of the graph 1008. In this example, the X-Axis represents the preventable cost in dollars of each member of the member cohort, and the Y-Axis represents the prior engagement of each member with other care management programs.

A tab 1016 included in the interface 1000 provides further statistics of the member cohort. For example, as shown, the tab 1016 includes information related to a total number of members in the member cohort, a total monthly costs of the cohort, a percentage of the cohort that is male, a percentage of the cohort that is less than 65 years old, and a number of emergency room utilizations per 1000 members. The information included in the tab 1016 is determined by the data analysis module 210 based on an analysis of the member data records of all members in the member cohort. Tab 1018 may be used to view a list of all members (e.g., identified by name or other member identifier) of the member cohort. Tab 1020 may be used to view all activities taken by care management staff with respect to the members of the member cohort. For example, the tab 1020 may include information related to members of the member cohort who have been contacted related to participation in a therapeutic intervention program.

### Modules, Components, and Logic

Certain embodiments are described herein as including logic or a number of components, modules, or mechanisms. Modules may constitute either software modules (e.g., code embodied on a machine-readable medium) or hardware modules. A “hardware module” is a tangible unit capable of performing certain operations and may be configured or arranged in a certain physical manner. In various example embodiments, one or more computer systems (e.g., a standalone computer system, a client computer system, or a server computer system) or one or more hardware modules of a computer system (e.g., a processor or a group of processors) may be configured by software (e.g., an application or application portion) as a hardware module that operates to perform certain operations as described herein.

In some embodiments, a hardware module may be implemented mechanically, electronically, or any suitable combination thereof. For example, a hardware module may include dedicated circuitry or logic that is permanently configured to perform certain operations. For example, a hardware module may be a special-purpose processor, such as a Field-Programmable Gate Array (FPGA) or an Application Specific Integrated Circuit (ASIC). A hardware module may also include programmable logic or circuitry that is temporarily configured by software to perform certain operations. For example, a hardware module may include software executed by a general-purpose processor or other programmable processor. Once configured by such software, hardware modules become specific machines (or specific components of a machine) uniquely tailored to perform the configured functions and are no longer general-purpose processors. It will be appreciated that the decision to implement a hardware module mechanically, in dedicated and permanently configured circuitry, or in temporarily configured circuitry (e.g., configured by software) may be driven by cost and time considerations.

Accordingly, the phrase “hardware module” should be understood to encompass a tangible entity, be that an entity that is physically constructed, permanently configured (e.g., hardwired), or temporarily configured (e.g., programmed) to operate in a certain manner or to perform certain operations described herein. As used herein, “hardware-implemented module” refers to a hardware module. Considering embodiments in which hardware modules are temporarily configured (e.g., programmed), each of the hardware modules need not be configured or instantiated at any one instance in time. For example, where a hardware module comprises a general-purpose processor configured by software to become a special-purpose process or, the general-purpose processor may be configured as respectively different special-purpose processors (e.g., comprising different hardware modules) at different times. Software accordingly configures a particular processor or processors, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple hardware modules exist contemporaneously, communications may be achieved through signal transmission (e.g., over appropriate circuits and buses) between or among two or more of the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions described herein. As used herein, “processor-implemented module” refers to a hardware module implemented using one or more processors.

Similarly, the methods described herein may be at least partially processor-implemented, with a particular processor or processors being an example of hardware. For example, at least some of the operations of a method may be performed by one or more processors or processor-implemented modules. Moreover, the one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., an API).

The performance of certain of the operations may be distributed among the processors, not only residing within a single machine, but deployed across a number of machines. In some example embodiments, the processors or processor-implemented modules may be located in a single geographic location (e.g., within a home environment, an office environment, or a server farm). In other example embodiments, the processors or processor-implemented modules may be distributed across a number of geographic locations.

### Example Machine Architecture and Machine-Readable

FIG. 11 is a block diagram illustrating components of a machine 1100, according to some example embodiments, able to read instructions from a machine-readable medium (e.g., a machine-readable storage medium) and perform any one or more of the methodologies discussed herein. Specifically, FIG. 11 shows a diagrammatic representation of the machine 1100 in the example form of a computer system, within which instructions 1116 (e.g., software, a program, an application, an applet, an app, or other executable code) for causing the machine 1100 to perform any one or more of the methodologies discussed herein may be executed. Additionally, or alternatively, the machine 1100 may correspond to any one of the client device 102, the web server 110, the application server 112, or the third-party computing system 118. The instructions transform the general, non-programmed machine into a particular machine programmed to carry out the described and illustrated functions in the manner described. In alternative embodiments, the machine 1100 operates as a standalone device or may be coupled (e.g., networked) to other machines. In a networked deployment, the machine 1100 may operate in the capacity of a server machine or a client machine in a server-client network environment, or as a peer machine in a peer-to-peer (or distributed) network environment. The machine 1100 may comprise, but not be limited to, a server computer, a client computer, a personal computer (PC), a tablet computer, a laptop computer, a netbook, a set-top box (STB), a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 1116, sequentially or otherwise, that specify actions to be taken by the machine 1100. Further, while only a single machine 1100 is illustrated, the term “machine” shall also be taken to include a collection of machines 1100 that individually or jointly execute the instructions 1116 to perform any one or more of the methodologies discussed herein.

The machine 1100 may include processors 1110, memory/storage 1130, and I/O components 1150, which may be configured to communicate with each other such as via a bus 1102. In an example embodiment, the processors 1110 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an ASIC, a Radio-Frequency Integrated Circuit (RFIC), another processor, or any suitable combination thereof) may include, for example, a processor 1112 and a processor 1114 that may execute the instructions 1116. The term “processor” is intended to include multi-core processor that may comprise two or more independent processors (sometimes referred to as “cores”) that may execute instructions contemporaneously. Although FIG. 11 shows multiple processors, the machine 1100 may include a single processor with a single core, a single processor with multiple cores (e.g., a multi-core processor), multiple processors with a single core, multiple processors with multiples cores, or any combination thereof.

The memory/storage 1130 may include a memory 1132, such as a main memory, or other memory storage, and a storage unit 1136, both accessible to the processors 1110 such as via the bus 1102. The storage unit 1136 and memory 1132 store the instructions 1116 embodying any one or more of the methodologies or functions described herein. The instructions 1116 may also reside, completely or partially, within the memory 1132, within the storage unit 1136, within at least one of the processors 1110 (e.g., within the processor's cache memory), or any suitable combination thereof, during execution thereof by the machine 1100. Accordingly, the memory 1132, the storage unit 1136, and the memory of the processors 1110 are examples of machine-readable media.

As used herein, “machine-readable medium” means a device able to store instructions and data temporarily or permanently, and may include, but is not limited to, random-access memory (RAM), read-only memory (ROM), buffer memory, flash memory, optical media, magnetic media, cache memory, other types of storage (e.g., Erasable Programmable Read-Only Memory (EEPROM)), and/or any suitable combination thereof. The term “machine-readable medium” should be taken to include a single medium or multiple media (e.g., a centralized or distributed database, or associated caches and servers) able to store the instructions 1116. The term “machine-readable medium” shall also be taken to include any medium, or combination of multiple media, that is cap able of storing instructions (e.g., instructions 1116) for execution by a machine (e.g., machine 1100), such that the instructions, when executed by one or more processors of the machine (e.g., processors 1110), cause the machine to perform any one or more of the methodologies described herein. Accordingly, a “machine-readable medium” refers to a single storage apparatus or device, as well as “cloud-based” storage systems or storage networks that include multiple storage apparatus or devices. The term “machine-readable medium” excludes signals per se.

Furthermore, the machine-readable medium is non-transitory in that it does not embody a propagating signal. However, labeling the tangible machine-readable medium “non-transitory” should not be construed to mean that the medium is incapable of movement—the medium should be considered as being transportable from one real-world location to another. Additionally, since the machine-readable medium is tangible, the medium may be considered to be a machine-readable device.

The I/O components 1150 may include a wide variety of components to receive input, provide output, produce output, transmit information, exchange information, capture measurements, and so on. The specific I/O components 1150 that are included in a particular machine will depend on the type of machine. For example, portable machines such as mobile phones will likely include a touch input device or other such input mechanisms, while a headless server machine will likely not include such a touch input device. It will be appreciated that the I/O components 1150 may include many other components that are not shown in FIG. 11. The I/O components 1150 are grouped according to functionality merely for simplifying the following discussion and the grouping is in no way limiting. In various example embodiments, the I/O components 1150 may include output components 1152 and input components 1154. The output components 1152 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 1154 may include alphanumeric input components (e.g., a keyboard, a touch screen configured to receive alphanumeric input, a photo-optical keyboard, or other alphanumeric input components), point based input components (e.g., a mouse, a touchpad, a trackball, a joy stick, a motion sensor, or another pointing instrument), tactile input components (e.g., a physical button, a touch screen that provides location and/or force of touches or touch gestures, or other tactile input components), audio input components (e.g., a microphone), and the like.

In further example embodiments, the I/O components 1150 may include biometric components 1156, motion components 1158, environmental components 1160, or position components 1162 among a wide array of other components. For example, the biometric components 1156 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram based identification), and the like. The motion components 1158 may include acceleration sensor components (e.g., accelerometer), gravitation sensor components, rotation sensor components (e.g., gyroscope), and so forth. The environmental components 1160 may include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detect concentrations of hazardous gases for safety or to measure pollutants in the atmosphere), or other components that may provide indications, measurements, or signals corresponding to a surrounding physical environment. The position components 1162 may include location sensor components (e.g., a Global Position System (GPS) receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 1150 may include communication components 1164 operable to couple the machine 1100 to a network 1180 or devices 1170 via a coupling 1182 and a coupling 1172, respectively. For example, the communication components 1164 may include a network interface component or other suitable device to interface with the network 1180. In further examples, the communication components 1164 may include wired communication components, wireless communication components, cellular communication components, Near Field Communication (NFC) components, Bluetooth® components (e.g., Bluetooth® Low Energy), Wi-Fi components, and other communication components to provide communication via other modalities. The devices 1170 may be another machine or any of a wide variety of peripheral devices (e.g., a peripheral device coupled via a Universal Serial Bus (USB)).

Moreover, the communication components 1164 may detect identifiers or include components operable to detect identifiers. For example, the communication components 1164 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF4110, Ultra Code, UCC RSS-2D bar code, and other optical codes), or acoustic detection components (e.g., microphones to identify tagged audio signals). In addition, a variety of information may be derived via the communication components 1064, such as location via Internet Protocol (IP) geo-location, location via Wi-Fi® signal triangulation, location via detecting an NFC beacon signal that may indicate a particular location, and so forth.

### Transmission Medium

In various example embodiments, one or more portions of the network 1180 may be an ad hoc network, an intranet, an extranet, a VPN, a LAN, a WLAN, a WAN, a WWAN, a MAN, the Internet, a portion of the Internet, a portion of the PSTN, a plain old telephone service (POTS) network, a cellular telephone network, a wireless network, a WiFi® network, another type of network, or a combination of two or more such networks. For example, the network 1180 or a portion of the network 1180 may include a wireless or cellular network and the coupling 1182 may be a Code Division Multiple Access (CDMA) connection, a Global System for Mobile communications (GSM) connection, or another type of cellular or wireless coupling. In this example, the coupling 1182 may implement any of a variety of types of data transfer technology, such as Single Carrier Radio Transmission Technology (1xRTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long range protocols, or other data transfer technology.

The instructions 1116 may be transmitted or received over the network 1180 using a transmission medium via a network interface device (e.g., a network interface component included in the communication components 1164) and using any one of a number of well-known transfer protocols (e.g., hypertext transfer protocol (HTTP)). Similarly, the instructions 1116 may be transmitted or received using a transmission medium via the coupling 1172 (e.g., a peer-to-peer coupling) to the devices 1170. The term “transmission medium” shall be taken to include any intangible medium that is capable of storing, encoding, or carrying the instructions 1116 for execution by the machine 1100, and includes digital or analog communications signals or other intangible media to facilitate communication of such software.

### Language

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Although an overview of the inventive subject matter has been described with reference to specific example embodiments, various modifications and changes may be made to these embodiments without departing from the broader scope of embodiments of the present disclosure. Such embodiments of the inventive subject matter may be referred to herein, individually or collectively, by the term “invention” merely for convenience and without intending to voluntarily limit the scope of this application to any single disclosure or inventive concept if more than one is, in fact, disclosed.

The embodiments illustrated herein are described in sufficient detail to enable those skilled in the art to practice the teachings disclosed. Other embodiments may be used and derived therefrom, such that structural and logical substitutions and changes may be made without departing from the scope of this disclosure. The Detailed Description, therefore, is not to be taken in a limiting sense, and the scope of various embodiments is defined only by the appended claims, along with the full range of equivalents to which such claims are entitled.

As used herein, the term “or” may be construed in either an inclusive or exclusive sense. Moreover, plural instances may be provided for resources, operations, or structures described herein as a single instance. Additionally, boundaries between various resources, operations, modules, engines, and data stores are somewhat arbitrary, and particular operations are illustrated in a context of specific illustrative configurations. Other allocations of functionality are envisioned and may fall within a scope of various embodiments of the present disclosure. In general, structures and functionality presented as separate resources in the example configurations may be implemented as a combined structure or resource. Similarly, structures and functionality presented as a single resource may be implemented as separate resources. These and other variations, modifications, additions, and improvements fall within a scope of embodiments of the present disclosure as represented by the appended claims. The specification and drawings are, accordingly, to be regarded in an illustrative rather than a restrictive sense.

In this document, the terms “a” or “an” are used, as is common in patent documents, to include one or more than one, independent of any other instances or usages of “at least one” or “one or more.” In the appended claims, the terms “including” and “in which” are used as the plain-English equivalents of the respective terms “comprising” and “wherein.” Also, in the following claims, the terms “including” and “comprising” are open-ended; that is, a system, device, article, or process that includes elements in addition to those listed after such a term in a claim are still deemed to fall within the scope of that claim. Moreover, in the following claims, the terms “first,” “ second,” “third,” and so forth are used merely as labels, and are not intended to impose numerical requirements on their objects.

What is claimed is:

1. A system comprising:
one or more processors of a machine;
a first machine-readable medium storing a plurality of member data records, each member data record including information about a member of a group;
a second machine-readable medium storing a plurality of data models previously generated by applying computer-implemented logistic regression techniques to the plurality of member data records, the plurality of data models including one or more risk models, a benefit data model, and a participation data model;
a third machine-readable medium storing instructions that, when executed by the one or more processors of the machine, cause the machine to perform operations comprising:
causing presentation, on a display of a client device, of a graphical user interface comprising one or more interactive elements, each of the one or more interactive elements enabling a user to specify a constraint for participation in a program;
receiving from the client device, one or more constraints specified using the one or more interactive elements;
identifying a set of candidate member data records from the plurality of member data records based on the one or more constraints, the set of candidate member data records corresponding to members qualifying for participation in the program;
determining by the one or more processors, a risk score associated with each candidate member data record using the one or more risk models, the risk score associated with each candidate member data record providing a measure of likelihood of the corresponding member to deviate from expected costs over a period of time by more than a threshold amount;
determining by the one or more processors, a benefit score associated with each candidate member data record using the benefit data model, the benefit score associated with each candidate member data record providing a measure of likelihood of the corresponding member to benefit from participation in the program, the benefit including a change to utilization of one or more services;
determining by the one or more processors, a participation score associated with each candidate member data record using the participation data model, the participation score associated with each candidate data member record providing a measure of a likelihood of the corresponding member to participate in the program;
identifying, from the set of candidate member data records, one or more target member data records based at least in part on a combination of respective risk scores, benefit scores, and participation scores of the set of candidate member data records, the one or more target member data records correspond to members to target for the program; and
updating the graphical user interface to present:
an indicator of a number of members to target for participation in the program;
a graph displaying information related to the one or more target member data records in graphical form, a type of information displayed in the graph being configurable by one or more graphical elements; and
a set of windows providing additional information related to the one or more target member data records, a first window from the set of windows including a statistical breakdown of aspects of the one or more target member data records, a second window including a list of the members to target for participation in the program, and a third window from the set of windows specifying one or more staff activities related to the one or more member data records.

2. The system of claim 1, wherein the operations further comprise matching target member data records to one or more staff member data records based on a comparison of the information included in the target member data records with data related to staff skills and staff availability.

3. The system of claim 1, wherein the operations further comprise accessing a subset of the plurality of member data records based on user constraints defining a member cohort, wherein the subset of the plurality of member data records corresponds to the member cohort, wherein the set of candidate member data records are identified from member data records corresponding to the member cohort.

4. The system of claim 1, wherein the identifying of the set of candidate member data records includes:
determining an expected cost value associated with each member data record in the plurality of member data records;
determining an actual cost value associated with each member data record;
calculating a delta value associated with each member data record, the delta value corresponding to a difference between the expected cost value and the actual cost value;
determining an average delta value for the plurality of member data records; and
identifying member data records as the candidate member data records based on a deviation of the delta values associated with the candidate member data records from the average delta value of the plurality of member data records.

5. The system of claim 1, wherein the determining of the participation score associated with each candidate member data record includes:
accessing, from the second machine-readable storage medium, the participation data model corresponding to the program, the participation data model comprising a plurality of participation metrics, each of the participation metrics corresponding to a factor that contributes to a likelihood of the member to participate in the program;
determining a value for each of the plurality of participation metrics based on the information included in the member data record;
weighting each participation metric according to a degree to which the participation metric contributes to the likelihood of the member to participate from the program; and
aggregating the weighted participation metric values to generate the participation score.

6. The system of claim 1, wherein the user interface further includes an indication of the benefit scores and participation scores of the target members.

7. The system of claim 1, wherein the benefit data model includes one or more benefit metrics that contribute to the likelihood of the member to benefit from participation in the program, wherein the one or more benefit metrics relate to at least one of: social factors or behavioral factors.

8. A method comprising:
causing presentation, on a display of a client device, of a graphical user interface comprising one or more interactive elements, each of the one or more interactive elements enabling a user to specify a constraint for participation in a program;
receiving from the client device, one or more constraints specified using the one or more interactive elements;
accessing from a first machine-readable storage medium member data comprising a plurality of member data records, each member data record including information about a member of a group;
identifying a set of candidate member data records from the plurality of member data records based on the one or more constraints, the set of candidate member data records corresponding to members qualifying for participation in the program;
accessing from a second machine-readable storage medium, a plurality of data models previously generated by applying computer-implemented logistic regression techniques to the plurality of member data records, the plurality of data models including one or more risk models, a benefit data model, and a participation data model;
determining a risk score associated with each candidate member data record using the one or more risk models, the risk score associated with each candidate member data record providing a measure of likelihood of the corresponding member to deviate from expected costs over a period of time by more than a threshold amount;
determining using one or more processors, a benefit score associated with each candidate member data record using the benefit data model, the benefit score associated with each candidate member data record providing a measure of a likelihood of the corresponding member to benefit from participation in the program, the benefit including a change to utilization of one or more services;
determining a participation score associated with each candidate member data record using the participation data model, the participation score associated with each candidate data member record providing a measure of likelihood of the corresponding member to participate in the program;
identifying, from the set of candidate member data records, one or more target member data records based on a combination of respective risk scores, benefit scores, and participation scores of the set of candidate member data records, the one or more target member data records corresponding to target members for participation in the program; and
updating the graphical user interface to present:
an indicator of a number of members to target for participation in the program;
a graph displaying information related to the one or more target member data records in graphical form, a type of information displayed in the graph being configurable by one or more graphical elements; and
a set of windows providing additional information related to the one or more target member data records, a first window from the set of windows including a statistical breakdown of aspects of the one or more target member data records, a second window including a list of the members to target for participation in the program, and a third window from the set of windows specifying one or more activities of staff related to the one or more member data records.

9. The method of claim 8, further comprising matching target member data records to one or more staff member data records based on a comparison of the information included in the target member data records with information included in a plurality of staff member data records.

10. The method of claim 9, wherein the matching of the target member data records to the one or more staff member data records is based on information related to staff availability and staff skills.

11. The method of claim 8, wherein the accessed member data corresponds to a member cohort defined by one or more user constraints.

12. The method of claim 8, wherein the identifying of the set of candidate member data records is based on a deviation of an actual cost value associated with each member data record from an expected cost value associated with each member data record.

13. The method of claim 8, wherein the identifying of the set of candidate member data records includes:
determining an expected cost value associated with each member data record in the plurality of member data records;
determining an actual cost value associated with each member data record;
calculating a delta value associated with each member data record, the delta value corresponding to a difference between the expected cost value and the actual cost value; and
identifying member data records as the candidate member data records based on the delta value associated with each member data record.

14. The method of claim 13, wherein the identifying of the set of candidate member data records further includes determining an average delta value for the plurality of member data records, wherein the identifying of the member data records as the candidate member data records is based on a deviation of the delta values associated with the candidate member data records from the average delta value of the plurality of member data records.

15. The method of claim 8, wherein the benefit data model includes one or more benefit metrics, the one or more benefit metrics including one or more factors that contribute to the likelihood of the member to benefit from participation in the program.

16. The method of claim 15, wherein the one or more benefit metrics relate to at least one of: social factors, or behavioral factors.

17. The method of claim 8, wherein:
the benefit data model corresponds to the program, the benefit data model comprising a plurality of benefit metrics, each of the benefit metrics corresponding to a factor that contributes to a likelihood of the member to benefit from participation in the program; and
the determining of the benefit score associated with each candidate member data record includes:
determining a value for each of the plurality of benefit metrics based on the information included in the member data record;
weighting each benefit metric according to a degree to which the benefit metric contributes to the likelihood of the member to benefit from participation in the program, the weighting of each benefit metric being determined from the benefit data model; and
aggregating the weighted benefit metric values to generate the benefit score.

18. The method of claim 8, wherein the participation data model includes one or more participation metrics including one or more factors that contribute to the likelihood of the member to participate in the program, the one or more factors including demographic factors and social factors.

19. The method of claim 8, wherein the likelihood to benefit includes a likelihood that participation in the program will result in a change to utilization of other services by the member such that actual costs associated with the member are reduced.

20. A non-transitory machine-readable storage medium embodying instructions that, when executed by at least one processor of a machine, cause the machine to perform operations comprising:
causing presentation, on a display of a client device, of a graphical user interface comprising one or more interactive elements, each of the one or more interactive elements enabling a user to specify a constraint for participation in a program;
receiving from the client device, one or more constraints specified using the one or more interactive elements;
accessing from a first machine-readable storage medium, member data comprising a plurality of member data records, each member data record including information about a member of a group;
identifying a set of candidate member data records from the plurality of member data records, the set of candidate member data records corresponding to members qualifying for participation in the program;
accessing from a second machine-readable storage medium, a plurality of data models previously generated by applying computer-implemented logistic regression techniques to the plurality of member data records, the plurality of data models including one or more risk models, a benefit data model, and a participation data model;
determining a risk score associated with each candidate member data record using one or more risk models, the risk score associated with each candidate member data record providing a measure of likelihood of the corresponding member to deviate from expected costs over a period of time by more than a threshold amount;
determining a benefit score associated with each candidate member data record using a benefit data model, the benefit score associated with each candidate member data record providing a measure of likelihood of the corresponding member to benefit from participation in the program, the benefit including a change to utilization of other services;
determining a participation score associated with each candidate member data record using the participation data model, the participation score associated with each candidate data member record providing a measure of a likelihood of the corresponding member to participate in the program;
identifying from the set of candidate member data records, one or more target member data records based on a combination of respective risk scores, benefit scores, and participation scores of the set of candidate member data records, the one or more target member data records correspond to members to target for participation in the program; and
updating the graphical user interface to present:
an indicator of a number of members to target for participation in the program;
a graph displaying information related to the one or more target member data records in graphical form, a type of information displayed in the graph being configurable by one or more graphical elements; and
a set of windows providing additional information related to the one or more target member data records, a first window from the set of windows including a statistical breakdown of aspects of the one or more target member data records, a second window including a list of the members to target for participation in the program, and a third window from the set of windows specifying one or more activities of staff related to the one or more member data records.
